(19) United States
(12) Patent Application Publication
(10) Pub. No.: US 2026/0236641 A1
(43) Pub. Date: Aug. 13, 2026

(54) IMPLEMENTATION OF HYBRID DENSITY FUNCTIONAL THEORY ON QUANTUM PROCESSORS WITH MOLLER-PLESSET PERTURBATION FOR CHEMICAL-COMPOUND SIMULATION

(71) Applicant: Tata Consultancy Services Limited, Mumbai (IN)

(72) Inventors: ANIRBAN MUKHERJEE, Mumbai (IN); ANANTHAKRISHNA GOPAL, Mumbai (IN); RITAM BANERJEE, Mumbai (IN)

(73) Assignee: Tata Consultancy Services Limited, Mumbai (IN)

(21) Appl. No.: 18/987,955

(22) Filed: Dec. 19, 2024

(30) Foreign Application Priority Data
Dec. 21, 2023 (IN) 202321087618

(51) Int. Cl. G06F 30/27; G16C 10/00

(52) U.S. Cl. CPC G06F 30/27; G16C 10/00

(57) ABSTRACT

Existing approaches provide limitations to scale Hybrid DFT calculations to large systems and there is no such scalable technique to correct errors during Hybrid DFT computation with tensors. This disclosure relates generally to implementation of Hybrid Density Functional Theory on Quantum Processors with Moller-Plesset Perturbation (MP2) corrected simulation. The system receives a plurality of inputs from a classical computer component to transmit to a quantum computer component. Initially, a density matrix is computed from a core Hamilton. Further, the quantum processor computes a direct (J) matrix, a Hybrid matrix, and a correlation exchange potentials to compute a Fock matrix. Further, the density matrix is iteratively performed for a criteria where difference between the density matrix and an updated density matrix converges to a value below a threshold convergence error. MP2 energy corrections are performed for extracting the plurality of properties associated with the chemical compound.

### PRIORITY CLAIM

This U.S. patent application claims priority under 35 U.S.C. § 119 to: Indian Patent Application number 202321087618, filed on Dec. 21, 2023. The entire contents of the aforementioned application are incorporated herein by reference.

### TECHNICAL FIELD

The disclosure herein generally relates to quantum processors, and, more particularly, to Implementation of hybrid Density Functional Theory on quantum processors with Moller-Plesset Perturbation for chemical-compound simulation.

### BACKGROUND

Quantum computers promises to solve industry-critical problems which are otherwise unsolvable or only very inefficiently addressable using classical computers. Key application areas include chemistry and materials, bioscience and bioinformatics, logistics, and finance. Interest in quantum computing has recently surged, in part due to a wave of advances in the performance of ready-to-use quantum computers. However, quantum computing platforms available have imposed significant constraints on simulations due to both the limited number of qubits available for the calculations as well as the relatively short qubit coherence times and modest gate fidelity. Therefore, a useful quantum computing application must be able to demonstrate superiority (i.e., quantum supremacy) over a classical simulation using relatively few qubits with a short depth.

Indeed, the applications of quantum algorithms for quantum chemistry remain limited in terms of size of affordable systems, as the size of the system dictates the number of required qubits. Even though the number of qubits on quantum devices is expected to increase rapidly, stable machines able to tackle real quantum chemistry systems are not expected in the next few years. In the near future, material and drug design may be aided by quantum computer assisted simulations. These have the potential to target chemical systems intractable by the most powerful classical computers. The enhancement of the compute power addresses a wide range of business problems related to discovery of novel materials in material sciences industry and novel drug molecules in pharma industry. It also addresses the modifications and robustness of thermodynamic properties of chemical systems i.e., novel materials or drugs as a functional of physical parameters like temperature, pressure, impurities, pH etc.

Existing quantum computing strategies include selecting a particular quantum algorithm and implementing it on a near term quantum device. Benchmarks of this character include using tensor networks, quantum communication protocols, and using a number of simple quantum circuits to benchmark the device. However, the resources offered by contemporary quantum computers are still limited, restricting the simulations to very simple molecules. In order to rapidly scale up to more interesting molecular systems.

Existing techniques cater to the limitations of near-term quantum device such as variational quantum eigen solver (VQE), quantum approximate optimization algorithm (QAOA) and variants, variational quantum linear systems solver, other quantum algorithms leveraging the variational principles, and quantum machine learning algorithms. In spite of such algorithmic innovations, many of these approaches have appeared to be impractical for commercially-relevant problems owing to their high cost in terms of number of measurements and runtime. Also, there is no such scalable technique to correct errors during Hybrid Density Functional Theory (DFT) computation with tensors.

In the conventional Kohn-sham DFT as stated above, the computational complexity scales cubically to the system size which is the consequence of the delocalized nature of the wave functions which are the eigen solutions of the Kohn Sham single particle Hamiltonian. To scale the Hybrid DFT calculations to large systems, there have been attempts to develop an algorithm which scales linearly with system size. One such code is ONETEP (order-N electronic total energy package) linear-scaling DFT calculations with large basis set (plane-wave) accuracy on parallel computers. It uses a basis of non-orthogonal generalized Wannier functions (NGWFs) expressed in terms of periodic cardinal sine (psinc) functions, which are in turn equivalent to a basis of plane-waves. ONETEP therefore is a combination of the benefits of linear scaling with a level of accuracy and variational bounds comparable to that of traditional cubic-scaling plane-wave approaches. During the calculation, the density matrix and the NGWFs are optimized with localization constraints. ONETEP optimizes the total energy of the system, ensuring self-consistent convergence of electronic structure.

Currently the efforts are towards speeding up the hybrid DFT calculations using Extra scale computing: CPU, GPU, MPI, Multiprocessing etc. However, all of these have memory and processing limitations. As quantum hardware and algorithms continue to develop, various industry sectors, particularly the pharmaceutical and material design domains, are applying quantum computation paradigm to their specific problems. There have been attempts made to implement DFT on a combination of classical and quantum processors, for example, US20220012382A1 which implements DFT on a classical processor and optimizes the DFT results on a quantum processor. However, the complex calculations are still performed on a classical processor which does not overcome the above mentioned bottlenecks in DFT calculations.

### SUMMARY

Embodiments of the present disclosure present technological improvements as solutions to one or more of the above-mentioned technical problems recognized by the inventors in conventional systems. For example, in one embodiment, a system for implementation of hybrid Density Functional Theory on quantum processors with Moller-Plesset Perturbation for chemical-compound simulation is provided. The system includes receiving via a one or more classical hardware processor a chemical compound to extract a plurality of properties and obtains a plurality of atomic coordinates of a plurality of molecular orbitals associated with a plurality of molecules comprised in the chemical compound. Further, determining via the one or more classical hardware processors, a plurality of inputs from the plurality of atomic coordinates comprising (i) a plurality of electron integrals, (i) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals. The plurality of inputs are transmitted from the one or more classical hardware processors of a classical computer to a one or more quantum processor of a quantum computer. The one or more quantum processors determines a density matrix from the core Hamiltonian.

Iteratively performing by the one or more quantum processor for a criteria, wherein the criteria specifies that a difference between the density matrix and an updated density matrix converges to a value below a threshold convergence error, wherein the steps of each iteration to validate the criteria comprises initially computing a direct (J) matrix from the density matrix on a second set of qubits and a first set of qubits in a quantum circuit. The quantum circuit comprises a (i) the first set of qubits, the second set of qubits, and a plurality of ancilla qubits. Initially, a Hybrid matrix (K) is computed using (i) a first Cholesky tensor of shape for a first Cholesky circuit component, (ii) a second Cholesky tensor of shape for a second Cholesky circuit component and (iii) the density matrix encoded with the second set of qubits including one control qubit and one target qubit to store coefficients, wherein Naux is a number of auxillary vectors and Nao is a number of atomic orbitals.

In one embodiment, the first Cholesky tensor of shape is given as (Naux, NaoNao). The second Cholesky tensor of shape is given as (Nao, NauxNao). The density matrix tensor shape is given as (Nao, Nao). The rectangular matrix tensor shape is given as (NauxNao, Nao).

Correlation exchange matrix is determined based on (i) the collocation matrix using a generalized gradient approximation (GGA), and (ii) the Hybrid matrix energy added for Density Functional Theory (DFT) computations. Further, Fock matrix is computed based on (i) the core Hamiltonian, (ii) the direct (J) matrix and the Hybrid (K) matrix constructed from the density matrix and the Cholesky tensor, and (iii) the correlation exchange matrix. Further, an updated density matrix is computed by performing qubitized diagonalization on the Fock matrix and a single particle rotation matrix C.

Further using the one or more quantum processors Moller-Plesser Perturbations (MP2) energy corrections is simulated on the updated density matrix and the Hybrid (K) matrix using the single particle rotation matrix C of the last convergence when the difference between the density matrix and an updated density matrix converges to a value above the threshold convergence error. Furthermore, the one or more quantum processors obtains (i) the quantum circuit, (ii) the updated density matrix, and (iii) the MP2 energy corrections to be performed by the one or more classical hardware processors for extracting the plurality of properties associated with the chemical compound.

In another aspect, a method implementation of hybrid Density Functional Theory on quantum processors with Moller-Plesset Perturbation for chemical-compound simulation is provided. The method includes receiving via a one or more classical hardware processor a chemical compound to extract a plurality of properties and obtains a plurality of atomic coordinates of a plurality of molecular orbitals associated with a plurality of molecules comprised in the chemical compound. Further, determining via the one or more classical hardware processors, a plurality of inputs from the plurality of atomic coordinates comprising (i) a plurality of electron integrals, (i) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals. The plurality of inputs are transmitted from the one or more classical hardware processors of a classical computer to a one or more quantum processor of a quantum computer. The one or more quantum processors determines a density matrix from the core Hamiltonian.

Iteratively the one or more quantum processor performs for a criteria, wherein the criteria specifies that a difference between the density matrix and an updated density matrix converges to a value below a threshold convergence error, wherein the steps of each iteration to validate the criteria comprises initially computing a direct (J) matrix from the density matrix on a second set of qubits and a first set of qubits in a quantum circuit. The quantum circuit comprises a (i) the first set of qubits, the second set of qubits, and a plurality of ancilla qubits. Initially, a Hybrid matrix (K) is computed using (i) a first Cholesky tensor of shape for a first Cholesky circuit component, (ii) a second Cholesky tensor of shape for a second Cholesky circuit component and (iii) the density matrix encoded with the second set of qubits including one control qubit and one target qubit to store coefficients, wherein Naux is a number of auxillary vectors and Nao is a number of atomic orbitals.

In one embodiment, the first Cholesky tensor of shape is given as (Naux, NaoNao). The second Cholesky tensor of shape is given as (Nao, NauxNao). The density matrix tensor shape is given as (Nao, Nao). The rectangular matrix tensor shape is given as (NauxNao, Nao).

Correlation exchange matrix is determined based on (i) the collocation matrix using a generalized gradient approximation (GGA), and (ii) the Hybrid matrix energy added for Density Functional Theory (DFT) computations. Further, Fock matrix is computed based on (i) the core Hamiltonian, (ii) the direct (J) matrix and the Hybrid (K) matrix constructed from the density matrix and the Cholesky tensor, and (iii) the correlation exchange matrix. Further, an updated density matrix is computed by performing qubitized diagonalization on the Fock matrix and a single particle rotation matrix C.

Further the one or more quantum processors computes Moller-Plesser Perturbations (MP2) energy corrections is simulated on the updated density matrix and the Hybrid (K) matrix using the single particle rotation matrix C of the last convergence when the difference between the density matrix and an updated density matrix converges to a value above the threshold convergence error. Furthermore, the one or more quantum processors obtains (i) the quantum circuit, (ii) the updated density matrix, and (iii) the MP2 energy corrections to be performed by the one or more classical hardware processors for extracting the plurality of properties associated with the chemical compound.

In yet another aspect, a non-transitory computer readable medium for implementation of hybrid Density Functional Theory on quantum processors with Moller-Plesset Perturbation for chemical-compound simulation is provided. The computer readable program, when executed on a system comprising one or more classical hardware processors communicably coupled to a one or more Quantum Processors via interfaces, causes the computing device to receive via a one or more classical hardware processor a chemical compound to extract a plurality of properties and obtains a plurality of atomic coordinates of a plurality of molecular orbitals associated with a plurality of molecules comprised in the chemical compound. Further, determining via the one or more classical hardware processors, a plurality of inputs from the plurality of atomic coordinates comprising (i) a plurality of electron integrals, (i) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals. The plurality of inputs are transmitted from the one or more classical hardware processors of a classical computer to a one or more quantum processor of a quantum computer. The one or more quantum processors determines a density matrix from the core Hamiltonian.

Iteratively the one or more quantum processor performs for a criteria, wherein the criteria specifies that a difference between the density matrix and an updated density matrix converges to a value below a threshold convergence error, wherein the steps of each iteration to validate the criteria comprises initially computing a direct (J) matrix from the density matrix on a second set of qubits and a first set of qubits in a quantum circuit. The quantum circuit comprises a (i) the first set of qubits, the second set of qubits, and a plurality of ancilla qubits. Initially, a Hybrid matrix (K) is computed using (i) a first Cholesky tensor of shape for a first Cholesky circuit component, (ii) a second Cholesky tensor of shape for a second Cholesky circuit component and (iii) the density matrix encoded with the second set of qubits including one control qubit and one target qubit to store coefficients, wherein Naux is a number of auxillary vectors and Nao is a number of atomic orbitals.

In one embodiment, the first Cholesky tensor of shape is given as (Naux, NaoNao). The second Cholesky tensor of shape is given as (Nao, NauxNao). The density matrix tensor shape is given as (Nao, Nao). The rectangular matrix tensor shape is given as (NauxNao, Nao).

Correlation exchange matrix is determined based on (i) the collocation matrix using a generalized gradient approximation (GGA), and (ii) the Hybrid matrix energy added for Density Functional Theory (DFT) computations. Further, Fock matrix is computed based on (i) the core Hamiltonian, (ii) the direct (J) matrix and the Hybrid (K) matrix constructed from the density matrix and the Cholesky tensor, and (iii) the correlation exchange matrix. Further, an updated density matrix is computed by performing qubitized diagonalization on the Fock matrix and a single particle rotation matrix C.

Further, the one or more quantum processors computes Moller-Plesser Perturbations (MP2) energy corrections is simulated on the updated density matrix and the Hybrid (K) matrix using the single particle rotation matrix C of the last convergence when the difference between the density matrix and an updated density matrix converges to a value above the threshold convergence error. Furthermore, the one or more quantum processors obtains (i) the quantum circuit, (ii) the updated density matrix, and (iii) the MP2 energy corrections to be performed by the one or more classical hardware processors for extracting the plurality of properties associated with the chemical compound.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 is a functional block diagram of a system (alternatively referred as Hybrid-Quantum classical system) for implementation of hybrid Density Functional Theory on quantum processors with Moller-Plesset Perturbation (MP2) corrected for chemical-compound simulation, in accordance with some embodiments of the present disclosure.

FIG. 2A, FIG. 2B and FIG. 2C (collectively referred as FIG. 2) is an exemplary flowchart of a method performed by the quantum computer of FIG. 1 implementing Hybrid Density Functional Theory (DFT) on quantum processors with Moller-Plesset Perturbation (MP2) corrected for chemical-compound simulation, in accordance with some embodiments of the present disclosure.

FIG. 3 illustrates a quantum circuit used to compute Hybrid (K) matrix in the method illustrated in FIG. 2, in accordance with some embodiments of the present disclosure.

FIG. 4 illustrates a quantum circuit used to compute correlation exchange potential (Vxc) in the method illustrated in FIG. 2, in accordance with some embodiments of the present disclosure.

FIG. 5 illustrates a quantum circuit performing Moller-Plesser Perturbations (MP2) energy corrections in the method illustrated in FIG. 2, in accordance with some embodiments of the present disclosure.

FIG. 6 illustrates a quantum circuit implementing Hybrid Density Functional Theory (DFT) on quantum processors with Moller-Plesset Perturbation (MP2) corrected for chemical-compound simulation in the method illustrated in FIG. 2, in accordance with some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Most industrially relevant chemical systems in the material sciences industry are bulk materials. One supercell made from a collection of neighboring unit cells involves around thousands of atoms which corresponds to several thousands of electronic orbitals even in the least correlated basis. Currently these chemical systems are treated within the Quantum Mechanical (QM) or Molecular Mechanical (MM) approach, where only a subsystem with strong quantum correlations is simulated using DFT and the rest is treated only via the MM technique. Identifying these subsystems requires doing prior MM and/or force field analysis that adds to additional overhead cost. The gap in applying this DFT technology to the complete system arises from the quartic and cubic scaling wall bottleneck i.e., for a 50000 electronic orbital system (corresponding to a supercell of the bulk material), computing even one KS-DFT step would require 0.001 secs on the FUGAKU—the world's second most powerful supercomputer with a Rmax of 442 PETA Flops. For all practical purposes, the DFT code should converge within 100 self-consistency steps therefore to simulate one large supercell on FUGAKU would require 0.1 seconds. For real world simulations of chemical compounds, one would require screening across several lakhs of molecular configurations in a supercell and that would require more than one day. The output electronic density computed from DFT needs to be in turn passed into Force-Field or MM modelling suites that then recomputes the geometric positioning of atoms and the DFT energies needs to be recomputed. Hence, for a bit larger system such calculations can enter several days of calculations even on the largest of the supercomputers. Similarly, the chemical systems in the Pharma industry that correspond to Protein-drug or Protein-Protein systems involve more than thousands of atoms which corresponds to a several thousands of electronic orbitals. These chemical systems are treated approximately within the QM or MM approach, only a subsystem is treated with strong quantum correlations and is simulated using DFT and the rest is treated only via the MM technique. Even the drug molecules in the pharma industry have more than 500 molecular weight, therefore screening across different drug molecules enters across several days of efforts.

DFT and Hybrid DFT although being a mature technology has a tremendous bottleneck. If an algorithm can reduce the time computational complexity of DFT for general chemical systems even nominally from cubic to quadratic or linear then several days of calculations can be reduced to a few days or within a day. This will enable faster and or more screening, handling larger QM regions and reduce the product discovery time drastically.

The present disclosure provides a method for implementing hybrid Density Functional Theory on quantum processors with Moller-Plesset Perturbation (MP2) for chemical-compound simulation. The system may be alternatively referred as Hybrid-Quantum classical system 100. Hybrid DFT is an extension of DFT which is an iterative method where calculations on classical platform are interweaved with calculations on quantum processors. Here, the diagonalization problem is mapped to a sequence of quadratic least squares problem using a Quantum linear systems. DFT provides an exact treatment of many-body quantum ground state given the exact exchange-correlation energy functional theory. The advantage of DFT on classical platforms is that only a small set of electron densities and non-interacting wavefunctions need to be stored and manipulated and can be stored efficiently.

Hybrid DFT is advantageous over other semi local DFT (LDA, GGA, meta GGA) because of 1. Reduction of self-interaction error due to addition of the exact exchange matrix (Hybrid (K) matrix contribution. It also provides betters band gaps, bond lengths, vibrational frequencies compared to semi-local DFT. Further, additional Moller-Plesset Perturbation (MP2) error wavefunction correction simulation adds the leading post-Hybrid DFT that betters the prediction of polarization, dipole moment and other physical properties important to assess the applicability of the chemical compound in different use-cases.

Kohn-Sham (KS) hybrid Density Functional Theory (DFT) process is initiated from a set of Np basis functions

$$𝔹 = {\{ {ϕ}_{μ} ( r ) \}}_{μ = 1}^{{N}_{b}}$$

to compute the core Hamiltonian h which is a N_{b}×N_{b} matrix. h constitutes the free particle energy of electrons and one-body nuclear potential arising from the electron-nucleus interactions, both these terms are independent of electronic density. Therefore h needs to be computed while initiating the self-consistent field (SCF) procedure. The direct (J) matrix, exact-exchange (K) matrix are electronic density dependent terms and are computed from the Electronic Repulsion Integral (ERI) tensor. The correlation exchange potential or the correlation exchange matrix (V^{xc}) is computed from the electronic density, its gradient and/or the Kinetic energy density. Together J, K and V^{xc} make the hybrid Kohn-Sham Fock Matrix functional F as given by Equation 1.

$$F [ D ] = h + J [ D ] - c K [ D ] + {V}^{x c} [ D ] \qquad Equation 1$$

In Equation 1, D is the one-particle density matrix discretized in the basis of {φ_{μ}} and c∈ [symbol] ⁺ is the modulation for the exact exchange term. The terms J, K can be computed from the AO-integral approach using the ERI tensor [symbol] ij|kl [symbol] and D as in Equations 2 and 3, respectively.

$${J}_{i j} = {\sum }_{k l} 〈 ij \vert kl 〉 {D}_{kl} \qquad Equation 2$$

$${K}_{i j} = {\sum }_{k l} 〈 ik \vert jl 〉 {D}_{kl} \qquad Equation 3$$

In Equations 2 and 3, the ERI tensor [symbol] ij|kl [symbol] is obtained by integrating the space of basis functions in [symbol] ³ according to Equation 4,

$$〈 ij \vert kl 〉 = \int {d}^{3} r {d}^{3} {r}^{′} {ϕ}_{i} ( r ) {ϕ}_{j} ( r ) \frac{1}{\vert r - {r}^{′} \vert} {ϕ}_{k} ( {r}^{′} ) {ϕ}_{l} ( {r}^{′} ) \qquad Equation 4$$

The correlation-exchange potential V^{xc} in the discretized basis {φᵢ} is computed by Equation 5 where the basis functions are defined according to Equation 6,

$${V}_{i j}^{x c} = {\int }_{{R}^{3}} {d}^{3} r {ϕ}_{i} ( r ) \frac{δ {E}^{x c} [ ρ ( r ) ]}{δρ ( r )} {ϕ}_{j} ( r ) \qquad Equation 5$$

$${ϕ}_{a} ( r ) = {( x - {A}_{x} )}^{{a}_{x}} {( 𝒴 - {A}_{𝒴} )}^{{a}_{𝒴}} {( z - {A}_{z} )}^{{a}_{z}} {e}^{( - {ϑ ( x - {A}_{x} )}^{2} )} {e}^{( - {ϑ ( z - {A}_{z} )}^{2} )} \qquad Equation 6$$

In Equation 6, Aₓ, A_{y}, A_{z} are nuclear coordinate locations of the Gaussian functions, a=(aₓ, a_{y}, a_{z}) are the integer cartesian quanta and ϑ is the cartesian Gaussian exponent. In Equation 5, E^{xc} is the exchange energy functional evaluated for the electronic density r and its form depends on the DFT method being used. The ERI tensor is represented using the Cholesky decomposition representation as given by Equation 7,

$$〈 ij \vert kl 〉 = {\sum }_{P} {L}_{i j}^{P} {L}_{k l}^{P} \qquad Equation 7$$

The Cholesky matrices L^{P} can be obtained from the Density-Fitting (DF) or the resolution of identity (RI) method as in Equation 8,

$${L}_{i j}^{P} = \frac{1}{\sqrt{{w}_{P}}} {\sum }_{Q} {u}_{Q P} 〈 ij \vert Q 〉 \qquad Equation 8$$

In Equation 8, [symbol] ij|Q [symbol] are 3-center 2-electron integrals represented in terms of the auxiliary basis functions

$${\{ {χ}_{P} ( r ) \}}_{P = 1}^{N a u x} ,$$

and u_{QP}, [symbol] _{P} are obtained from the spectral decomposition of 2-center 2-electron integrals

$${V}_{P Q} = {\sum }_{R} {u}_{P R} {w}_{R} {u}_{Q R}^{*}$$

represented in the auxiliary basis. 3-center 2-electron integrals are given by Equation 9 and V_{PQ}= [symbol] P|Q [symbol] represents two center two electron integrals given by Equation 10.

$$〈 ij \vert P 〉 = \int {d}^{3} r {d}^{3} {r}^{′} \frac{{ϕ}_{i} ( r ) {ϕ}_{j} ( r ) {Λ}_{P} ( {r}^{′} )}{\vert r - {r}^{′} \vert} \qquad Equation 9$$

$$〈 P \vert Q 〉 = \int {d}^{3} r \frac{{Λ}_{P} ( r ) {Λ}_{Q} ( r )}{\vert r - {r}^{′} \vert} \qquad Equation 10$$

The J and K matrix elements are computed using the RI approach as in Equation 11.

$${J}_{i j} = {\sum }_{P} {L}_{i j}^{P} {\sum }_{k l} {L}_{k l}^{P} {D}_{k l} , {K}_{ij} = {\sum }_{pl} {L}_{il}^{P} {\sum }_{k} {L}_{j k}^{P} {D}_{k l} \qquad Equation 11$$

When the KS-DFT is classically computed, the complexity of computing J and K is O(pN²) and this complexity resides in computing K. Computing h and V^{xc} has lower complexity of O(N²). Next step of DFT is to solve the generalized eigenvalue problem for the Fock matrix accounting for the overlap between basis functions Sᵢⱼ= [symbol] φᵢ|φⱼ [symbol] and the density matrix D^{l} of a current iteration as given by Equation 12.

$$F [ {D}^{( l )} ] {C}^{( l + 1 )} = {SC}^{( l + 1 )} {\hat{E}}^{( l + 1 )} \qquad Equation 12$$

In Equation 12, the one particle density matrix D^{(l)}=C^{(l)}WC^{(l)†}, where the occupancy matrix Wᵢⱼ=θ(μ−Eᵢ)δᵢⱼ fills up Nₑ/2 lowest energy levels below the chemical potential μ.

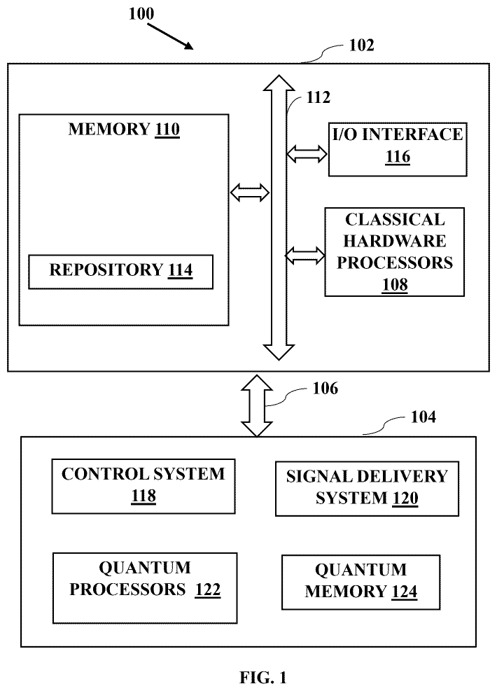

Referring now to the drawings, and more particularly to FIG. 1 through FIG. 6, where similar reference characters denote corresponding features consistently throughout the figures, there are shown preferred embodiments, and these embodiments are described in the context of the following exemplary system and/or method.

FIG. 1 is a functional block diagram of a system (alternatively referred as Hybrid-Quantum classical system) for implementation of hybrid Density Functional Theory on quantum processors with Moller-Plesset Perturbation (MP2) corrected for chemical-compound simulation, in accordance with some embodiments of the present disclosure.

The Hybrid-Quantum classical system 100 includes a classical computing system 102, a quantum computing system 104 and a communication interface 106. The classical computing system 102 comprises classical hardware processors 108, at least one memory such as a memory 110, a I/O interface 116. The classical hardware processors 108, the memory 110, and the Input/Output (I/O) interface 116 may be coupled by a system bus such as a system bus 112 or a similar mechanism. In an embodiment, the classical hardware processors 108 can be one or more hardware processors. The classical hardware processors and the hardware processors is interchangeably used throughout the document. Similarly, the classical computing system is a normal computing system.

The I/O interface 116 may include a variety of software and hardware interfaces, for example, a web interface, a graphical user interface, and the like, for example, interfaces for peripheral device(s), such as a keyboard, a mouse, an external memory, a printer, and the like. Further, the I/O interface 116 may enable the system 100 to communicate with other devices, such as web servers, and external databases. The I/O interface 116 can facilitate multiple communications within a wide variety of networks and protocol types, including wired networks, for example, local area network (LAN), cable, etc., and wireless networks, such as Wireless LAN (WLAN), cellular, or satellite. For the purpose, the I/O interface 116 may include one or more ports for connecting several computing systems with one another or to another server computer.

The one or more hardware processors 108 may be implemented as one or more microprocessors, microcomputers, microcontrollers, digital signal processors, central processing units, node machines, logic circuitries, and/or any devices that manipulate signals based on operational instructions. Among other capabilities, the one or more hardware processors 108 is configured to fetch and execute computer-readable instructions stored in the memory 110.

The memory 110 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

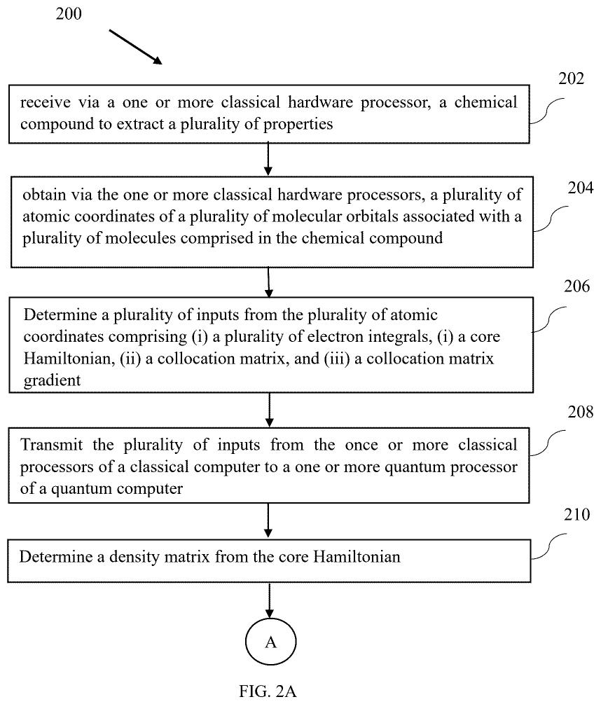

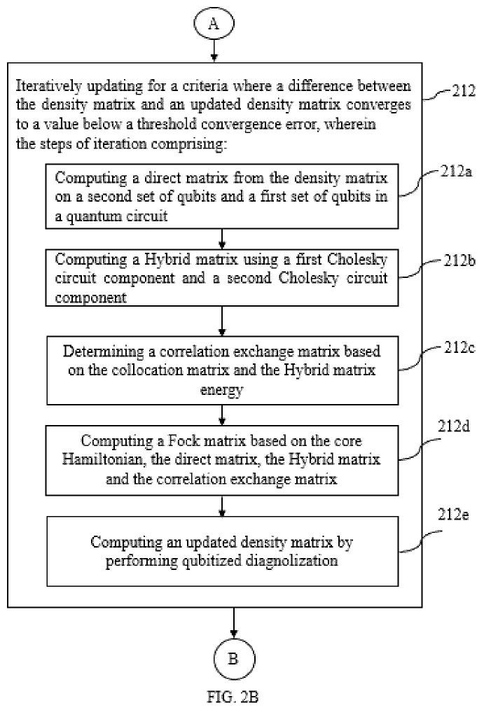

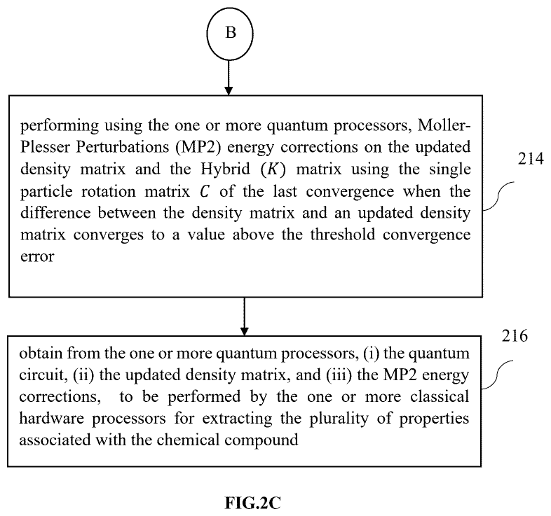

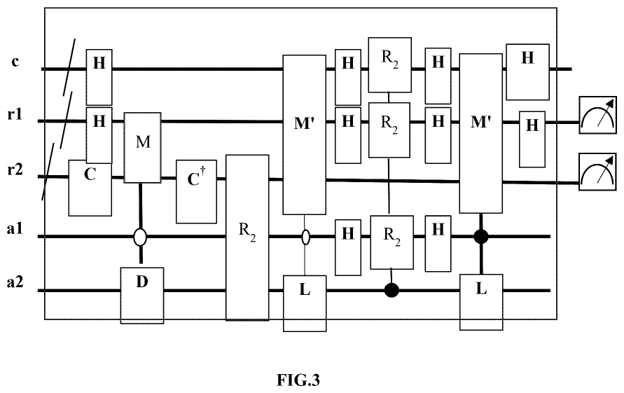

In an embodiment, the memory 110 includes a data repository 114. The data repository (or repository) 114 may include a plurality of abstracted piece of code for refinement and data that is processed, received, or generated as a result of the execution of the method illustrated in FIG. 2 and FIG. 3. Although the data repository 114 is shown internal to the system 100, it should be noted that, in alternate embodiments, the data repository 114 can also be implemented external to the system 100, where the data repository 114 may be stored within a database (repository 114) communicatively coupled to the system 100. The data contained within such external database may be periodically updated. For example, new data may be added into the database (not shown in FIG. 1) and/or existing data may be modified and/or non-useful data may be deleted from the database. In one example, the data may be stored in an external system, such as a Lightweight Directory Access Protocol (LDAP) directory and a Relational Database Management System (RDBMS).

The example quantum computing system 104 shown in FIG. 1 includes a control system 118, a signal delivery system 120, a one or more Quantum Processors 122 and a quantum memory 124. The quantum computing system 104 may include additional or different features, and the components of a quantum computing system may operate as described with respect to FIG. 1 or in another manner.

The exemplified quantum computing system 104 shown in FIG. 1 can perform quantum computational tasks (such as, for example, quantum simulations or other quantum computational tasks) by executing quantum algorithms. In some implementations, the quantum computing system 104 can perform quantum computation by storing and manipulating information within individual quantum states of a composite quantum system. For example, Qubits (i.e., Quantum bits) can be stored in and represented by an effective two-level sub-manifold of a quantum coherent physical system in the one or more Quantum Processors 122.

In an embodiment, the quantum computing system 104 can operate using gate-based models for quantum computing. For example, the Qubits can be initialized in an initial state, and a quantum logic circuit comprised of a series of quantum logic gates can be applied to transform the qubits and extract measurements representing the output of the quantum computation. The exemplified Quantum Processors 122 shown in FIG. 1 may be implemented, for example, as a superconducting quantum integrated circuit that includes Qubit devices. The Qubit devices may be used to store and process quantum information, for example, by operating as ancilla Qubits, data Qubits or other types of Qubits in a quantum algorithm. Coupler devices in the superconducting quantum integrated circuit may be used to perform quantum logic operations on single qubits or conditional quantum logic operations on multiple qubits. In some instances, the conditional quantum logic can be performed in a manner that allows large-scale entanglement within the one or more Quantum Processors 122. Control signals may be delivered to the superconducting quantum integrated circuit, for example, to manipulate the quantum states of individual Qubits and the joint states of multiple Qubits. In some instances, information can be read from the superconducting quantum integrated circuit by measuring the quantum states of the qubit devices. The one or more Quantum Processors 122 may be implemented using another type of physical system.

The exemplified one or more Quantum Processors 122, and in some cases all or part of the signal delivery system 120, can be maintained in a controlled cryogenic environment. The environment can be provided, for example, by shielding equipment, cryogenic equipment, and other types of environmental control systems. In some examples, the components in the one or more Quantum Processors 122 operate in a cryogenic temperature regime and are subject to very low electromagnetic and thermal noise. For example, magnetic shielding can be used to shield the system components from stray magnetic fields, optical shielding can be used to shield the system components from optical noise, thermal shielding and cryogenic equipment can be used to maintain the system components at controlled temperature, etc.

In the example shown in FIG. 1, the signal delivery system 120 provides communication between the control system 118 and the one or more Quantum Processors 122. For example, the signal delivery system 120 can receive control signals from the control system 118 and deliver the control signals to the one or more Quantum Processors 122. In some instances, the signal delivery system 120 performs preprocessing, signal conditioning, or other operations to the control signals before delivering them to the Quantum Processors 122. In an embodiment, the signal delivery system 120 includes connectors or other hardware elements that transfer signals between the Quantum Processors 122 and the control system 118. For example, the connection hardware can include signal lines, signal processing hardware, filters, feedthrough devices (e.g., light-tight feedthroughs, etc.), and other types of components. In some implementations, the connection hardware can span multiple different temperature and noise regimes. For example, the connection hardware can include a series of temperature stages that decrease between a higher temperature regime (e.g., at the control system 118) and a lower temperature regime (e.g., at the Quantum Processors 122).

In the example quantum computer system 104 shown in FIG. 1, the control system 118 controls operation of the Quantum Processors 122. The exemplified control system 118 may include data processors, signal generators, interface components and other types of systems or subsystems. Components of the example control system 118 may operate in a room temperature regime, an intermediate temperature regime, or both. For example, the control system 118 can be configured to operate at much higher temperatures and be subject to much higher levels of noise than are present in the environment of the Quantum Processors 122. In some embodiments, the control system 118 includes a classical computing system that executes software to compile instructions for the Quantum Processors 122. For example, the control system 118 may decompose a quantum logic circuit or quantum computing program into discrete control operations or sets of control operations that can be executed by the hardware in the Quantum Processors 122. In some embodiments the control system 118 applies a quantum logic circuit by generating signals that cause the Qubit devices and other devices in the Quantum Processors 122 to execute operations. For instance, the operations may correspond to single-Qubit gates, two-Qubit gates, Qubit measurements, etc. The control system 118 can generate control signals that are communicated to the Quantum Processors 122 by the signal delivery system 120, and the devices in the Quantum Processors 122 can execute the operations in response to the control signals.

In some other embodiments, the control system 118 includes one or more classical computers or classical computing components that produce a control sequence, for instance, based on a quantum computer program to be executed. For example, a classical processor may convert a quantum computer program to an instruction set for the native gate set or architecture of the Quantum Processors 122. In some cases, the control system 118 includes a microwave signal source (e.g., an arbitrary waveform generator), a bias signal source (e.g., a direct current source) and other components that generate control signals to be delivered to the Quantum Processors 122. The control signals may be generated based on a control sequence provided, for instance, by a classical processor in the control system 118. The example control system 118 may include conversion hardware that digitizes response signals received from the Quantum Processors 122. The digitized response signals may be provided, for example, to a classical processor in the control system 118.

In some embodiments, the quantum computer system 104 includes multiple quantum information processors that operate as respective quantum processors. In some cases, each Quantum Processors can operate independent of the others. For instance, the quantum computer system 104 may be configured to operate according to a distributed quantum computation model, or the quantum computer system 104 may utilize multiple Quantum Processors in another manner.

In some implementations, the quantum computer system 104 includes multiple control systems, and each Quantum Processors may be controlled by a dedicated control system. In some implementations, a single control system can control multiple Quantum Processors; for instance, the control system 118 may include multiple domains that each control a respective Quantum Processors. In some instances, the quantum computing system 104 uses multiple Quantum Processors to execute multiple unentangled quantum computations (e.g., multiple Variational Quantum Eigen solver (VQE)) that collectively simulate a single quantum mechanical system.

In an embodiment, the quantum memory 124 is a quantum-mechanical version of classical computer memory. The classical computer memory stores information such as binary states and the quantum memory 124 stores a quantum state for later retrieval. These states hold useful computational information known as Qubits. In an embodiment, the communication interface 106 which connects the classical computing system 102 and the quantum computing system 104 is a high speed digital interface.

FIG. 2A, FIG. 2B and FIG. 2C (collectively referred as FIG. 2) is an exemplary flowchart of a method performed by the quantum computer of FIG. 1 implementing Hybrid Density Functional Theory (DFT) on quantum processors with Moller-Plesset Perturbation (MP2) corrected for chemical-compound simulation, in accordance with some embodiments of the present disclosure.

In an embodiment, the system 100 includes one or more data storage devices or the memory 110 operatively coupled to the one or more hardware processor(s) 108 and is configured to store instructions for execution of steps of the method 200 by the one or more hardware processors 108. The steps of the method 200 of the present disclosure will now be explained with reference to the components or blocks of the system 100 as depicted in FIG. 1, the steps of flow diagram as depicted in FIG. 2 and the quantum circuit of FIG. 3. The method 200 may be described in the general context of computer executable instructions. Generally, computer executable instructions can include routines, programs, objects, components, data structures, procedures, modules, functions, etc., that perform particular functions or implement particular abstract data types. The method 200 may also be practiced in a distributed computing environment where functions are performed by remote processing devices that are linked through a communication network. The order in which the method 200 is described is not intended to be construed as a limitation, and any number of the described method blocks can be combined in any order to implement the method 200, or an alternative method. Furthermore, the method 200 can be implemented in any suitable hardware, software, firmware, or combination thereof.

Now referring to FIG. 2, at step 202 of the method 200, one or more classical hardware processors are configured to receive a chemical compound to extract a plurality of properties.

The chemical compound may be at least one of a molecule or a solid. Molecule here refers to a general arrangement of atoms, and the solid here refers to a crystalline form that corresponds to a periodic arrangement of atoms. For example, when it comes to Pharma Industry the drug capsule for instance is a drug crystal, the atoms have a periodic arrangement. In the pharma industry, the chemical compound may be a drug lead and its conformations whose physical properties must be analyzed to determine feasibility of synthesizing the drug from the drug lead. The plurality of properties of drug lead includes large HOMO-LUMO gap in solvent medium, acid-base dissociation constant (pKa) of a drug, lower dipole moment lesser than 10.

At step 204 of the method 200 the one or more classical hardware processors are configured to obtain a plurality of atomic coordinates of a plurality of molecular orbitals associated with a plurality of molecules comprised in the chemical compound.

At step 206 of the method 200, the one or more classical hardware processors are configured to determine a plurality of inputs from the plurality of atomic coordinates comprising (i) a plurality of electron integrals, (i) a core Hamiltonian, (ii) a collocation matrix, and (iii) a collocation matrix gradient. The plurality of electron integrals comprises a 3-center 2-electron integrals, 2-center 2-electron integrals, and 2-center 1-electron integrals.

Each electron integral describe the Coulomb repulsion integral that are computed in atomic orbital basis and can be represented in either spherical or cartesian coordinates. The electron integral describes the overlap between the basis states. The plurality of electron integrals are determined using tools such as LIBCINT, Python-based Simulations of Chemistry Framework (PySCF), NorthWest computational Chemistry (NWchem) etc. The collocation matrix is a rectangular matrix of dimensions (Ng, Nao), wherein Ng represents a number of real space grid points, and Nao is a number of basis functions. It comprises a plurality of basis functions of a plurality of atomic orbitals and a plurality of points on numerical grid. Each of the plurality of basis functions is a Gaussian wave function centered around the atomic coordinates.

Further, at step 208 of the method 200 are configured to transmit the plurality of inputs from the one or more classical hardware processors of a classical computer to a one or more quantum processor of a quantum computer.

The plurality of inputs determined from each molecular orbits are transmitted to the quantum processors of the quantum computer.

Now referring to the step 210 of the method 200, the one or more quantum processors determines a density matrix from the core Hamiltonian. One example way of diagonalizing the core Hamiltonian is described in Indian patent application number 202321061415.

Once the density matrix is determined at step 212 of the method 200, the one or more quantum processors iteratively perform for a criteria where a difference between the density matrix and an updated density matrix converges to a value below a threshold convergence error. The convergence criteria is said to be satisfied when norm of a difference between the updated density matrix at a current iteration and the density matrix at a previous iteration is below the threshold convergence error. The threshold convergence error may be for example 10⁻⁹. Steps 212a to 212e are performed at each iteration to update the density matrix. At step (212a), a direct density matrix (alternatively referred to as J matrix, Coulomb matrix etc.) is computed from the density matrix on a second set of qubits and a first set of qubits in a quantum circuit component of a quantum circuit. The quantum circuit comprises a (i) the first set of qubits, the second set of qubits, and a plurality of ancilla qubits. As a first component in the quantum circuit the density matrix is loaded. The quantum circuit component is defined for local density approximation given in Equation 13,

$${V}_{J} = {H}^{⊗ m} {V}_{L}^{′} R {V}_{L} {H}^{⊗ m} O ( D ) \qquad Equation 13$$

Referring now FIG. 3, at step 212b a Hybrid matrix (alternatively referred as K matrix) is computed using (i) a first Cholesky tensor of shape (Naux, NaoNao) for a first Cholesky circuit component, (ii) a second Cholesky tensor of shape (Nao, NauxNao) for a second Cholesky circuit component and (iii) the density matrix encoded with the second set of qubits including one control qubit and one target qubit to store coefficients. Here, Naux is a number of auxillary vectors and Nao is a number of atomic orbitals. The quantum circuit is further loaded with the first Cholesky circuit component and the first Cholesky circuit component as inputs. In an embodiment, the quantum circuit is mathematically represented as: log Naux+2 log Nao+2 qubits. The first set of qubits is referred as log Naux and the second set of qubits is referred as 2 log Nao. Additional 2 qubits one among them is control and the other is ancillary qubit.

In one embodiment, the first Cholesky tensor of shape is given as (Naux, NaoNao). The second Cholesky tensor of shape is given as (Nao, NauxNao). The density matrix tensor shape is given as (Nao, Nao). The rectangular matrix tensor shape is given as (NauxNao, Nao).

Initially, the computed direct (J) matrix and the density matrix are loaded in the quantum circuit. The density matrix is loaded on the first set of 2n=2 log Nao qubits in the quantum circuit of m+2n+1=log Naux+2 log Nao+1 qubits. Where, Nao=Number of atomic orbitals, Naux=Number of auxillary vectors in the Density fitting or Resolution of identity approach). The Density matrix is loaded using its Eigen decomposition representation that involves,
1.

$$( \begin{matrix}Nao \\ 2\end{matrix} )$$

Given rotations on log Nao qubits.
2. Then block encoding a diagonal matrix of 1's in the initial Ne/2 diagonal bocks and zeros in the rest using 2 log Nao qubits.
3. Another set of

$$( \begin{matrix}Nao \\ 2\end{matrix} )$$

Givens rotation with conjugate angles on Ne/2 diagonals. (This is a state-preparation oracle for the quantum circuit) as given in Equation 14,

$$Equation 14$$

$${V}_{D} = {I}_{2}^{⊗ m} ⊗ [ ( C ⊗ {I}_{2}^{⊗ n} ⊗ {I}_{2}^{{a}_{1}} ) ( {\sum }_{k} \vert k , k , 0 〉 〈 k , k , 0 \vert ⊗ ( θ ( μ - {E}_{k} ) {I}_{{a}_{1}} + ( 1 - θ ( μ - {E}_{k} ) ) {Y}_{{a}_{1}} ) + \vert k , k , 1 〉 〈 k , k , 1 \vert ⊗ {I}_{2} ) ( {C}^{†} ⊗ {I}_{2}^{⊗ n} ⊗ {I}_{2}^{{a}_{1}} ) ] ] ⊗ {I}_{{a}_{2}}$$

Note that the density matrix D is encoded with 2 log Nao+1 control qubits (the additional control qubit is the a1 qubit) and the coefficients are stored on the target qubit a2 this is encoded is differently than the encoding O(D), where the encoding takes on target qubit a1 and the control qubits are 2 log N (a2 qubit is free no operations happen on it). With the above block encoding the Density matrix readouts are obtained as in Equation 15,

$$〈 P , i , 0 , 0 , 0 , 0 \vert {V}_{D} \vert P , j , 0 , 0 , 0 , 0 〉 = {\sum }_{k = 1}^{{N}_{e} / 2} {C}_{i k} {C}_{k j}^{*} = {D}_{i j} \qquad Equation 15$$

$$Where {N}_{e} = 2 {\sum }_{k = 1}^{N} θ ( μ - {E}_{k} )$$

Further, as a next component to be loaded on the quantum circuit a first Cholesky matrix is encoded on the first set of qubits and the second set of qubits with a first Cholesky tensor shape (NauxNao, Nao) using a sequence of Multi-controlled gates to form a Cholesky circuit component. The first Cholesky matrix is created using the 3-center 2-electron integrals, 2-center 2-electron integrals on a CPU, where the control is on the first set of qubits and the data is loaded on the ancilla qubits. On the Quantum Processor or on the Quantum simulator as a next component of the Quantum Circuit the Cholesky matrix is created from the 3-center 2-electron and 2-center 2-electron integrals as represented in Equation 8, on the quantum processing units. That is a (Nao, Nao, Naux) electronic repulsion integral tensor which we encode on the same log Naux+2 log Nao+2 qubits using the sequence of multi-controlled gates on the Quantum Processors. The controls are on the log Naux+2 log Nqubits and the data is loaded on the ancilla qubits. This is qubitized block encoding of the classical rectangular matrix of dimension (NauxNao, Nao). The density matrix of dimension (Nao, Nao) multiplies the first Cholesky Matrix encoded as (NauxNao, Nao) to generate another intermediate matrix of dimension (NauxNao, Nao) as given in Equation 16,

$${V}_{L} {R}_{2} {V}_{D} \qquad Equation 16$$

where VL is the Quantum circuit block that encodes the Cholesky tensor and R3 is the diffusion operator that mixes all the configurations in the combined space of the Auxillary basis Naux and the Atomic basis Nao. It is to be noted from the first multiplication in the case of the J matrix between the unitary operations that encoded the Cholesky matrix and the Density matrix as given in Equation 17, that was VLO(D), in that case no diffusion was involved.

$$Equation 17$$

$$O ( D ) = {I}_{2}^{⊗ m} ⊗ [ ( C ⊗ {I}_{2}^{⊗ n} ⊗ {I}_{2}^{{a}_{1}} ) ( {\sum }_{k} \vert k , k 〉 〈 k , k \vert ⊗ ( θ ( μ - {E}_{k} ) {I}_{a 1} + ( 1 - θ ( μ - {E}_{k} ) ) {Y}_{a 1} ) ( {C}^{†} ⊗ {I}_{2}^{⊗ n} ⊗ {I}_{2}^{{a}_{1}} ) ] ⊗ {I}_{a 2}$$

On the Quantum Processors or on the Quantum simulator, the second Cholesky circuit component is encoded by a second Cholesky matrix with a second Cholesky tensor shape (Nao, NauxNao) on the first set of qubits, the second set of qubits using the sequence of Multi-controlled gates to form a second Cholesky circuit component. The control is on the first set of qubits and the data is loaded on ancilla qubits. The first quantum circuit component is composed by multiplying the first Cholesky circuit component with the first Cholesky tensor shape (NauxNao, Nao), and the second Cholesky circuit component is composed with the transpose of the second Cholesky tensor shape (Nao, NauxNao) and the intermediate matrix to obtain sequences of bitstrings as given in Equation 18 and Equation 19,

$$〈 0 , i , 0 , 0 , 0 \vert {H}^{⊗ m} {V}_{L}^{′} {R}_{2} {V}_{L} {R}_{2} {V}_{D} {H}^{⊗ m} \vert 0 , j , 0 , 1 , 0 〉 = \sum {L}_{ij}^{a} {L}_{kl}^{a} {D}_{jk} = {K}_{ij} \qquad Equation 18$$

$$Where ,$$

$$Equation 19$$

$${V}_{L}^{′} = {\sum }_{c = 0. {r}_{1} = 0 , {r}_{2} = 0}^{{N}_{aux} - 1 , {N}_{ao} - 1} [ \vert c , {r}_{1} , {r}_{2} , 0 〉 〈 c , {r}_{1} , {r}_{2} , 0 \vert ⊗ ( {L}_{{r}_{1} {r}_{2}}^{{c}^{′}} I + i \sqrt{1 - {( {L}_{{r}_{1} {r}_{2}}^{{c}^{′}} )}^{2} Y} ) + \vert c , {r}_{1} , {r}_{2} , 1 〉 〈 c , r , 1 \vert ⊗ {I}_{2} ]$$

Further, Hybrid matrix is computed by diffusing the first Cholesky circuit component and the second Cholesky circuit component with a diffusion operator Rectangular matrix and the intermediate matrix. On the Quantum Processors or on the Quantum simulator (referring to an example in later embodiments an Isometry free proof for computing Hybrid matrix is illustrated) the second diffusion is between the two Cholesky happens in the combined space of the first set of qubits and the second set of qubits that the enables the multiplication of the rectangular matrix representation of the Cholesky tensor with shape (Nao, NaoNaux) and the intermediate matrix (NauxNao, Nao). This multiplication is different between the first Cholesky matrix and the second Cholesky matrix that happened only across the Naux auxiliary basis direction for the J matrix. In that case the operation on the Quantum circuit was carried out by the diffusion R that acted only the qubits associated with the auxiliary basis. By measuring the second set qubits in the log Naux+2 log Nao+2 qubits to obtain all sequences of bitstrings to read entries of the Direct matrix. The rows and columns are marked from the bitstrings configuration. At this step, the bitstrings measurements are repeated depending on the overlap between the plurality of states to perform amplitude amplification to enhance probability of the sequences of bitstrings.

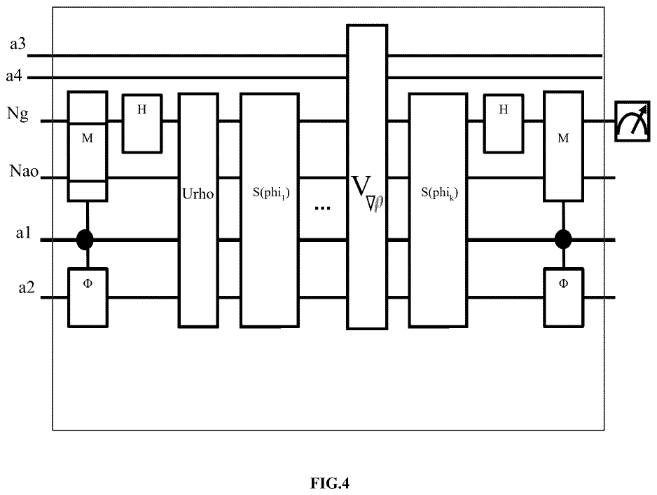

Once the Hybrid matrix is computed, at step 212c (referring now FIG. 4) a correlation exchange matrix is determined based on (i) the collocation matrix using a generalized gradient approximation (GGA), and (ii) the Hybrid matrix energy added for DFT computations. Here, the basis functions for different numerical grid points comprise the collocation matrix. Here Ng is the number of real spaces grid points and Nao is the number of basis functions. The quantum circuit is constructed using Hadamard and multi-controlled gates to encode the collocation matrix.

Once the correlation exchange is computed, at step 212d a Fock matrix is computed based on (i) the core Hamiltonian, (ii) the direct (J) matrix and the Hybrid (K) matrix constructed from the density matrix and the Cholesky tensor, and (iii) the correlation exchange matrix. The Fock matrix is being composed an additional ancilla qubit is used to add the K matrix contribution as given in Equation 20,

$$Equation 20$$

$${U}_{F} = \vert 00 〉 〈 00 \vert ⊗ {V}_{h} + \vert 01 〉 〈 01 ⊗ [ {H}^{⊗ m} {V}_{L}^{′} {RV}_{L} O ( D ) {H}^{⊗ m} ] + \vert 11 〉 〈 11 \vert ⊗ [ {H}^{⊗ m} {V}_{L}^{′} {R}_{2} {V}_{L} {R}_{2} {D}_{D} {H}^{⊗ m} ] + \vert 10 〉 〈 10 \vert ⊗ {H}^{⊗ ng} {V}_{Φ} {V}_{Z}$$

Then, at step 212e an updated density matrix is computed by performing qubitized diagonalization on the Fock matrix and a single particle rotation matrix C.

Further referring to the step 214 of the method 200, the one or more quantum processors performs Moller-Plesser Perturbations (MP2) energy corrections on the updated density matrix and the Hybrid (K) matrix using the single particle rotation matrix C of the last convergence when the difference between the density matrix and an updated density matrix converges to a value above the threshold convergence error.

MP2 correction simulator is enabled, firstly by computing the direct matrix (J), the Hybrid (K) matrix and the correlation exchange (Vxc) potentials from the plurality of electron integrals and the density matrix using the Quantum circuits as discussed earlier. Secondly, this enable construction of the Fock matrix, which is diagonalized using a quantum circuit (Qubitized diagonalization) to create the single particle rotation matrix C. Thirdly these rotations matrix C is used along with electron occupancies in the orbitals (2 when both electrons with up and down spin occupied or 0 when not occupied) to create the current step density matrix that is compared with last step density matrix by computing the norm of the difference of density matrices. Further, if the norm is above tolerance then the first two steps (J, K, Vxc construction) and the diagonalization process leading to the next density matrix is continued otherwise the density convergence continues.

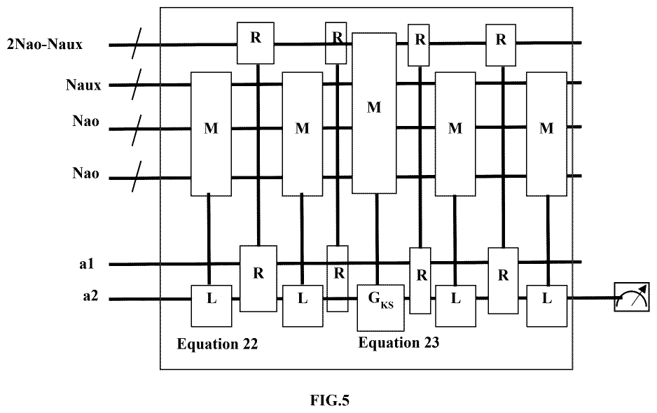

Referring now to FIG. 5, Moller-Plesser Perturbations (MP2) energy corrections are performed on the updated density matrix and the Hybrid (K) matrix based on the converges threshold convergence error. MP2 simulator obtains the Hybrid matrix, the first set of qubits, the second set of qubits and two copies of single particle unitary rotation matrix (C) as inputs. The MP2 energy correction is given by Equation 21,

$$Equation 21$$

$${E}_{C}^{M P 2} = {\sum }_{ijab} \frac{〈 ia \vert jb 〉 ( 2 〈 ia \vert jb 〉 - 〈 ja \vert ib 〉 )}{{ϵ}_{i} + {ϵ}_{j} - {ϵ}_{a} - {ϵ}_{b}} = {\sum }_{pij , qab} \frac{{L}_{ia}^{p} {L}_{jb}^{p} ( 2 {L}_{ia}^{q} {L}_{jb}^{q} - {L}_{ja}^{q} {L}_{ib}^{q} )}{{ϵ}_{i} + {ϵ}_{j} - {ϵ}_{a} - {ϵ}_{b}}$$

The MP2 correction is a wavefunction correction that is added to the DFT Energy. To begin with two copies of the single particle unitary rotation matrix (C) computed from the last Hybrid DFT step is applied on the two sets of log Nao, log Nao qubits, respectively. The first Cholesky tensor shape (Naux, Nao, Nao) is encoded with the first set of qubits and the second set of qubits for reshaping. Starting from the C Given rotations obtained from the Kohn-Sham diagonalization (FC=SCE) of Hybrid DFT post-convergence is completed, the single particle rotation matrix C is obtained and Molecular orbital energy eigenvalues, ϵ1, ϵnorbs here Nao=2norbs. Then, perform a set of Givens rotation to map the Atomic basis functions φa (x, y, z) to the Kohn-Sham Molecular Orbital basis and encode the Cholesky in the Kohn Sham Orbital basis as given in Equation 22,

$$Equation 22$$

$${V}_{L}^{′} = {\sum }_{c = 0. {r}_{1} = 0 , {r}_{2} = 0}^{{N}_{aux} - 1 , {N}_{ao} - 1} [ {I}^{⊗ p} ⊗ C ⊗ C ⊗ {I}_{2} \vert c , {r}_{1} , {r}_{2} , 0 〉 〈 c , {r}_{1} , {r}_{2} , 0 \vert ⊗ {I}^{⊗ p} ⊗ {C}^{†} ⊗ {C}^{†} ⊗ {I}_{2} ⊗ {I}_{2} + {I}^{⊗ p} ⊗ C ⊗ C ⊗ {I}_{2} \vert c , {r}_{1} , {r}_{2} , 1 〉 〈 c , {r}_{1} , {r}_{2} , 1 \vert {I}^{⊗ p} ⊗ C ⊗ C ⊗ {I}_{2} ⊗ ( {L}_{{r}_{1} {r}_{2}}^{{c}^{′}} I + i \sqrt{1 - {( {L}_{{r}_{1} {r}_{2}}^{{c}^{′}} )}^{2} Y} ) ]$$

The Cholesky tensor is composed by first placing the Unitary circuit for the Cholesky matrix then the Diffusion operator R then another Cholesky circuit. Once the first Cholesky tensor is reshaped, the reshaped first Cholesky tensor is loaded into the quantum circuit. The above step is repeated then one more time the two copies of the single particle rotation matrices C and C acts on the two subsets of log Nao qubits each. Next, the energy denominator comprises the Kohn Sham orbital energies obtained from convergence point. Then the energy denominator comprising of four single particle Fock energies as E1-E2-E3+E4 is encoded on the quantum circuit as given in Equation 23,

$$Equation 23$$

$${G}_{KS} = {\sum }_{rc} [ \vert i , j , k , l , 0 〉 〈 i , j , k , l , 0 \vert ⊗ ( \frac{1}{{ϵ}_{i} - {ϵ}_{j} + {ϵ}_{k} - {ϵ}_{l}} I + \sqrt{1 - \frac{1}{( {ϵ}_{i} - {ϵ}_{j} + {ϵ}_{k} - {ϵ}_{l}^{2} )}} Y ) ] + \vert i , j , k , l , 1 〉 〈 i , j , k , l , 1 \vert ⊗ {I}_{2}$$

Further, a pair of updated first Cholesky tensor is obtained to reiterate the loop to compute the Hybrid matrix leading to the MP2 correction being encoded on the Quantum circuit. The other component of the MP2 corrections are obtained by encoding two Cholesky tensors followed by the Block encoding of the denominator and then again followed by the two Cholesky tensors being encoded in the reverse order is given in Equation 24,

$$Equation 24$$

$$〈 0 , 0 , 0 , 0 , 0 \vert {H}^{⊗ ( 4 {logN}_{ao} )} {V}_{L}^{′} {RV}_{L} {G}_{KS} {V}_{L} {RV}_{L}^{′} {H}^{⊗ ( 4 {logN}_{ao} )} \vert 0 , 0 , 0 , 0 , 0 〉 = {\sum }_{pij , qab} {L}_{ia}^{p} {L}_{jb}^{p} \frac{1}{{ϵ}_{i} + {ϵ}_{j} - {ϵ}_{a} - {ϵ}_{b}} {L}_{ja}^{q} {L}_{ib}^{q}$$

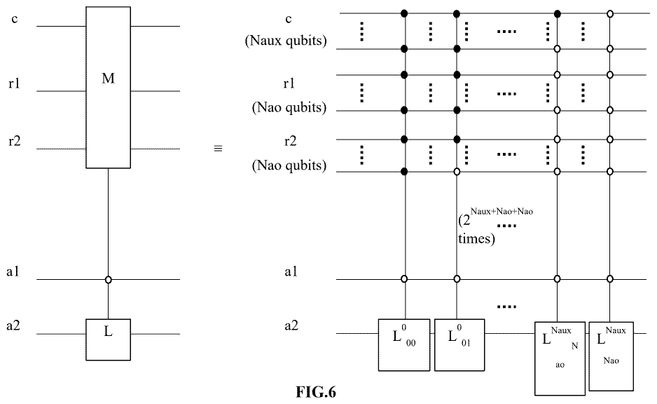

Referring now FIG. 6, finally at step 216 of the method 200, the one or more classical hardware processors obtain from the one or more quantum processors, (i) the quantum circuit, (ii) the updated density matrix, and (iii) the MP2 energy corrections, for extracting the plurality of properties associated with the chemical compound.

Example 1: For selecting cathode materials for rechargeable batteries, accurate optimized geometry of the cathode molecules in electrolyte environment, and associated ground state energy are calculated from method 200. This will then be used to compute more accurate energy enthalpy differences for the redox reactions. In turn the energy enthalpy differences are used to calculate high open cell voltages (OCV) (equilibrium voltage). A higher OCV leads to a higher cut-off for the recharging voltage. Cathode materials with different co-doped transition metal oxides (like Co, Ni, Mn) are screened using high OCV as the deciding factor. As there are a large number of properties to optimize: Number of battery recharging cycles, Charging percentage, time to recharge, weight of battery material. The number of battery materials number in more than a million, to screen them we need faster quantum chemistry calculation based on Density functional theory.

Example 2: In order to attain carbon neutrality in the near future, novel capture technologies have to be discovered. Carbon dioxide adsorption on amine polymers is one such technology. To assess the amount of carbon dioxide that can be adsorbed the BET surface area is computed from classical Molecular dynamics, but it requires calculating the potential energy surface (PES) from electronic calculations. Calculating an accurate PES with post-DFT corrections is computationally expensive which can be overcome by the method 200 disclosed herein.

Example 3: For drug conformational search and drug design, the molecular descriptors, conformations, solvation energy is obtained from the method 200 disclosed herein. Then from the conformational energies and the descriptors the subset of drug molecule structure and conformations with desired target properties are prepared via high throughput experimentation.

Experimental Results: The Hybrid matrix computation was performed on quantum processor for the inputs received from one or more classical hardware processors using method 200. The classical complexity is measured in terms of space and time complexity. Similarly, the quantum complexity is measured in terms of number of qubits and gate complexity. The results are recorded in Table 1.

TABLE 1
Implemenation of Hybrid DFT

| | | | Quantum Complexity | |
|---|---|---|---|---|
| | Classical Complexity | | | Gate |
| Quantity | Space | Time | No. of Qubits | complexity |
| J(AO) | $O ( {N}_{b}^{4} )$ | $O ( {N}_{b}^{4} )$ | 4 log N_{b} + 2 | 8 log N_{b} |
| J(DF) | $O ( {N}_{b}^{2} {N}_{aux} )$ | $O ( {N}_{b}^{2} {N}_{aux} )$ | 2 log N_{b} + log Nₐᵤₓ | 4 log N_{b} + 2 log Nₐᵤₓ |
| J(sN) | $O ( {N}_{b}^{2} {N}_{g} )$ | $O ( {N}_{b}^{2} {N}_{g} )$ | 2 log N_{b} + log N_{g} | 4 log N_{b} + 2 log N_{g} |
| K(AO) | $O ( {N}_{b}^{4} )$ | $O ( {N}_{b}^{4} )$ | 4 log N_{b} + 2 | 8 log N_{b} |
| K(DF) | $O ( {N}_{b}^{2} {N}_{aux} )$ | $O ( {N}_{b}^{3} {N}_{aux} )$ | 2 log Nb + log Nₐᵤₓ | 4 log N_{b} + 2 log Nₐᵤₓ |
| K(sN) | $O ( {N}_{bg}^{2} {N}_{aux} )$ | $O ( {N}_{b}^{2} {N}_{g} )$ | log N_{b} + log N_{g} + 2 | 2 log N_{b} + 2 log N_{g} |

Isometry free proof for computing Hybrid matrix-On the Quantum Processors or on the Quantum simulator is illustrated. The second diffusion is between the two Cholesky happens in the combined space of the first set of qubits and the second set of qubits that the enables multiplication of the rectangular matrix representation of the Cholesky tensor with shape (Nao, NaoNaux) and the intermediate matrix (NauxNao, Nao).

For example if A and B are general rectangular matrices of dimensions dim(A)=(N, P) and dim(B)=(P, M) then there is a unitary operation U(A, B) of dimension 2ⁿ^{_{q}}*2ⁿ^{_{q}} that operates on a system of n_{q}=p+max(m, n)+2 qubit registers|· [symbol] p|· [symbol] max(m, n)|· [symbol] a1|· [symbol] a2 where n=┌log₂ N┐, m=┌log₂ M┐, p=[log₂ P]) and block encodes the matrix multiplication of A and B is represent in Equation A1,

$$Equation A 1$$

$${{〈 0 {\vert}_{p} 〈 i {\vert}_{max ( m , n )} {〈 0 {\vert}_{{a}_{1}} {〈 0 {\vert}_{{a}_{2}} U ( A , B ) \vert 0 〉}_{p} \vert j 〉}_{max ( m , n )} \vert 1 〉}_{{a}_{1}} \vert 0 〉}_{{a}_{2}} = \frac{1}{max ( M , N ) P} \vert \frac{{\sum }_{k} {A}_{ik} {B}_{kj}}{‖ A ‖ ‖ B ‖} .$$

Proof is illustrated from Equation (A2-A9), Let us consider the normalized

$$matrices {A}^{′} = A / ( √ 2 ‖ A ‖ ) ,$$

$${B}^{′} = B / ( √ 2 ‖ B ‖ )$$

For the above normalized matrices, two unitary operators V(A) is given in Equation A2 and V(B) is given in Equation A3,

$$Equation A2$$

$${V}_{A} = {\sum }_{c = 0 , r = 0}^{{2}^{p} , {2}^{max ( m , n )}} [ \vert c , r , 0 〉 〈 c , r , 0 \vert ⊗ ( {A}_{rc}^{′} I + i \sqrt{1 - {A}_{rc}^{′ 2}} Y ) + \vert c , r , 1 〉 〈 c , r , 1 \vert ⊗ {I}_{2} ]$$

$$Equation A3$$

$${V}_{B} = {\sum }_{c = 0 , r = 0}^{{2}^{p} , {2}^{max ( m , n )}} [ \vert c , r , 0 〉 〈 c , r , 0 \vert ⊗ {I}_{2} + \vert c , r , 1 〉 〈 c , r , 1 \vert ⊗ ( {B}_{cr}^{′} I + i \sqrt{1 - {B}_{cr}^{′ 2}} Y ) ]$$

The classical data of the B matrix is loaded in the quantum circuit using the state preparation oracle V_{B}H^{⊗p} on the initial state |0 [symbol] |j [symbol] |1 [symbol] |0 [symbol] as given in Equation A4,

$$Equation A4$$

$$\vert {Φ}_{B} 〉 = {V}_{B} {H}^{⊗ p} \vert 0 〉 \vert j 〉 \vert 1 〉 \vert 0 〉 = \frac{1}{\sqrt{P}} {\sum }_{c} [ {B}_{cj}^{′} \vert c , j , 1 , 0 〉 + \sqrt{1 - {B}_{cj}^{′ 2}} \vert c , j , 1 , 1 〉 ]$$

The classical data of the A matrix is loaded in the quantum circuit using the state preparation oracle V_{A}H^{⊗p} as given in Equation A5,

$$Equation A 5$$

$$\vert {Φ}_{A} 〉 = {V}_{A} {H}^{⊗ p} \vert 0 〉 \vert i 〉 \vert 0 〉 \vert 0 〉 = \frac{1}{\sqrt{P}} {\sum }_{c} [ {A}_{ic}^{′} \vert c , i , 0 , 0 〉 + \sqrt{1 - {A}_{ic}^{′ 2}} \vert c , i , 0 , 1 〉 ]$$

Note that the states |Φ_{A} [symbol] and |Φ_{B} [symbol] are orthogonal as given in Equation A6,

$$〈 {Φ}_{A} \vert {Φ}_{B} 〉 = 0 \qquad Equation A 6$$

Further, a diffusion operator R acting on the row registers and the ancillas a₁, a₂ and R is given in Equation A7,

$$Equation A7$$

$$R = {I}_{2}^{⊗ p} ⊗ [ {H}^{⊗ max ( m , n )} ⊗ H ⊗ {I}_{2} ) ( 2 \vert 0 , 0 , 0 〉 〈 0 , 0 , 0 \vert - 1 ) {H}^{⊗ max ( m , n )} ⊗ H ⊗ {I}_{2} ) ] = {I}_{2}^{⊗ p} ⊗ [ {\sum }_{k , l} 2 \frac{\vert k , + , 0 〉 〈 l , + , 0 \vert}{max ( M , N )} - I ]$$

Then the overlap between these two states |Φ_{A} [symbol] and R|Φ_{B} [symbol] is given by [symbol] Φ_{A}|R|Φ_{B} [symbol] in Equation A8,

$$Equation A8$$

$$〈 {Φ}_{A} \vert R \vert {Φ}_{B} 〉 = 〈 0 , i , 0 , 0 \vert {H}_{c}^{⊗ p} {V}_{A}^{†} {RV}_{B} {H}_{c}^{⊗ p} \vert 0 , j , 1 , 0 〉 = \frac{2 {\sum }_{c} {A}_{ic}^{′} {B}_{cj}^{′}}{max ( M , N ) P} = \frac{{\sum }_{c} {A}_{ic} {B}_{cj}}{max ( M , N ) P ‖ A ‖ ‖ B ‖}$$

It is proven with the construction that existence of U(A, B) that can be defined without any isometry as given in Equation A9,

$$U ( A , B ) = {H}_{c}^{⊗ p} {V}_{A}^{†} {RV}_{B} {H}_{c}^{⊗ p} \qquad Equation A9$$

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem of quantum computing. The embodiment, thus provides implementation of hybrid density functional theory on quantum processors with Moller-Plesset Perturbation (MP2) corrected for chemical-compound simulation. Moreover, the embodiments herein further provides Hybrid DFT is advantageous over other semi local DFT (LDA, GGA, meta GGA) because of reduction of self-interaction error due to addition of the exact exchange matrix (K-matrix). The proposed Quantum simulation method provides betters band gaps, bond lengths, vibrational frequencies compared to semi-local DFT. MP2 wavefunction correction adds the leading post-DFT correction that betters the prediction of polarization, dipole moment and other physical properties important to assess the applicability of a chemical compound in different use cases.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means, and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A Hybrid-Quantum simulation method, performed by a system comprising one or more classical hardware processors and a one or more quantum processors, wherein the one or more classical hardware processors are communicably coupled to the one or more quantum processors by respective interfaces, wherein the quantum simulation method comprising:
receiving via the one or more classical hardware processors, a chemical compound to extract a plurality of properties;
obtaining via the one or more classical hardware processors, a plurality of atomic coordinates of a plurality of molecular orbitals associated with a plurality of molecules comprised in the chemical compound;
determining via the one or more classical hardware processors, a plurality of inputs from the plurality of atomic coordinates comprising (i) a plurality of electron integrals, (ii) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals;
transmitting the plurality of inputs from the one or more classical hardware processors of a classical computer to a one or more quantum processor of a quantum computer;
determining by the one or more quantum processors, a density matrix from the core Hamiltonian;
iteratively performing, by the one or more quantum processors, until a criteria is satisfied, wherein the criteria specifies that a difference between the density matrix and an updated density matrix converges to a value below a threshold convergence error, wherein the steps of each iteration to validate the criteria comprises:
(i) computing, a direct (J) matrix from the density matrix on a second set of qubits and a first set of qubits and loading the direct (J) matrix in a quantum circuit, wherein the quantum circuit comprises a (i) the first set of qubits, the second set of qubits, and a plurality of ancilla qubits;
(ii) computing a Hybrid matrix (K) using (i) a first Cholesky tensor of shape for a first Cholesky circuit component, (ii) a second Cholesky tensor of shape for a second Cholesky circuit component, and (iii) the density matrix encoded with the second set of qubits including one control qubit and one target qubit to store coefficients, wherein Naux is a number of auxillary vectors and Nao is a number of atomic orbitals, wherein in the quantum circuit the first Cholesky circuit component and the first Cholesky circuit component are loaded as inputs;
(iii) determining a correlation exchange matrix based on (i) the collocation matrix using a generalized gradient approximation (GGA), and (ii) the Hybrid (K) matrix energy added for DFT computations;
(iv) computing an Fock matrix based on (i) the core Hamiltonian, (ii) the direct (J) matrix and the Hybrid (K) matrix constructed from the density matrix and the Cholesky tensor, and (iii) the correlation exchange matrix; and
(v) computing an updated density matrix by performing qubitized diagonalization on the Fock matrix and a single particle rotation matrix C; and
performing using the one or more quantum processors, Moller-Plesser Perturbations (MP2) energy corrections on the updated density matrix and the Hybrid (K) matrix using the single particle rotation matrix C of the last convergence when the difference between the density matrix and an updated density matrix converges to a value above the threshold convergence error; and
obtaining from the one or more quantum processors, (i) the quantum circuit, (ii) the updated density matrix, and (iii) the MP2 energy corrections, to be performed by the one or more classical hardware processors for extracting the plurality of properties associated with the chemical compound.

2. The Hybrid-Quantum simulation method of claim 1, wherein the Hybrid (K) matrix is computed by performing the steps of:
obtaining the direct (J) matrix and the density matrix;
encoding a first Cholesky matrix on (i) a first set of qubits and (ii) a second set of qubits with a first Cholesky tensor shape using a sequence of Multi-controlled gates to form a Cholesky circuit component, wherein the first Cholesky matrix is created using the 3-center 2-electron integrals, 2-center 2-electron integrals on a CPU, where the control is on the first set of qubits and the data is loaded on the ancilla qubits;
encoding a second Cholesky matrix with a second Cholesky tensor shape on the first set of qubits, the second set of qubits using the sequence of Multi-controlled gates to form a second Cholesky circuit component, where the control is on the first set of qubits and the data is loaded on ancilla qubits;
composing a first quantum circuit component by multiplying the first Cholesky circuit component with the first Cholesky tensor shape;
composing a second Cholesky circuit component with the transpose of the second Cholesky tensor shape and the intermediate matrix to obtain sequences of bitstrings;
computing the Hybrid matrix by diffusing the first Cholesky circuit component and the second Cholesky circuit component with a diffusion operator Rectangular matrix and the Intermediate matrix;
measuring the second set qubits to obtain all sequences of bitstrings to read entries of the direct matrix; and
performing amplitude amplification to enhance probability of the sequences of bitstrings by removing overlap between a plurality of states.

3. The Hybrid-Quantum simulation method of claim 1, wherein the intermediate matrix is computed from the first Cholesky matrix by multiplying the density matrix tensor shape with a rectangular matrix tensor shape.

4. The Hybrid-Quantum simulation method of claim 1, wherein the MP2 energy corrections are performed on the updated density matrix and the Hybrid matrix using the single particle rotation matrix C of the last convergence by performing the steps of:
obtaining the Hybrid matrix, the first set of qubits, the second set of qubits and two copies of single particle unitary rotation matrix (C);
encoding the first Cholesky tensor shape with the first set of qubits and the second set of qubits to reshape the first Cholesky tensor;
loading the reshaped first Cholesky tensor into the quantum circuit;
repeating the single particle unitary rotation matrix to generate subsets of the first set of qubits;
encoding on the first quantum circuit energy denominator comprising of four single particle Fock energies; and
obtaining a pair of updated first Cholesky tensor to reiterate the loop to compute the Hybrid matrix.

5. A Hybrid-Quantum system for implementation of hybrid density functional theory on Quantum Processors, comprising:
one or more classical hardware processors operatively coupled to the at least one memory storing programmed instructions;
one or more Quantum Processors operatively coupled to at least one Quantum Memory storing programmed instructions;
a quantum-classical interface communicably coupling the one or more classical hardware processors of a computing system with one or more Quantum Processors of a quantum computer;
one or more Input/Output (I/O) interfaces;
wherein the one or more Quantum processors and the one or more classical hardware processors are configured by the programmed instructions to:
receive via a one or more classical hardware processor, a chemical compound to extract a plurality of properties;
obtain via the one or more classical hardware processors, a plurality of atomic coordinates of a plurality of molecular orbitals associated with a plurality of molecules comprised in the chemical compound;
determine via the one or more classical hardware processors, a plurality of inputs from the plurality of atomic coordinates comprising (i) a plurality of electron integrals, (i) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals;
transmit the plurality of inputs from the one or more classical hardware processors of a classical computer via the quantum-classical interface to a one or more quantum processor of a quantum computer;
determine by the one or more quantum processors, a density matrix from the core Hamiltonian;
iteratively perform by the one or more quantum processor, for a criteria, wherein the criteria specifies that a difference between the density matrix and an updated density matrix converges to a value below a threshold convergence error, wherein the steps of each iteration to validate the criteria comprises:
compute a direct (J) matrix from the density matrix on a second set of qubits and a first set of qubits in a quantum circuit, wherein the quantum circuit comprises a (i) the first set of qubits, the second set of qubits, and a plurality of ancilla qubits;
compute a Hybrid matrix (K) using (i) a first Cholesky tensor of shape for a first Cholesky circuit component, (ii) a second Cholesky tensor of shape for a second Cholesky circuit component, and (iii) the density matrix encoded with the second set of qubits including one control qubit and one target qubit to store coefficients, wherein in the quantum circuit the first Cholesky circuit component and the first Cholesky circuit component are loaded as inputs;
determine a correlation exchange matrix based on (i) the collocation matrix using a generalized gradient approximation (GGA), and (ii) the Hybrid matrix energy added for DFT computations;
compute an Fock matrix based on (i) the core Hamiltonian, (ii) the direct (J) matrix and the Hybrid (K) matrix constructed from the density matrix and the Cholesky tensor, and (iii) the correlation exchange matrix; and
compute an updated density matrix by performing qubitized diagonalization on the Fock matrix and a single particle rotation matrix C; and
perform using the one or more quantum processors, Moller-Plesser Perturbations (MP2) energy corrections on the updated density matrix and the Hybrid (K) matrix using the single particle rotation matrix C of the last convergence when the difference between the density matrix and an updated density matrix converges to a value above the threshold convergence error; and
obtain from the one or more quantum processors, (i) the quantum circuit, (ii) the updated density matrix, and (iii) the MP2 energy corrections, to be performed by the one or more classical hardware processors for extracting the plurality of properties associated with the chemical compound.

6. The Hybrid-Quantum system as claimed in claim 5, wherein the Hybrid (K) matrix is computed by performing the steps of:
(i) obtaining the direct (J) matrix and the density matrix;
(ii) encoding a first Cholesky matrix on (i) a first set of qubits and (ii) a second set of qubits with a first Cholesky tensor shape using a sequence of Multi-controlled gates to form a Cholesky circuit component, wherein the first Cholesky matrix is created using the 3-center 2-electron integrals, 2-center 2-electron integrals on a CPU, where the control is on the first set of qubits and the data is loaded on the ancilla qubits;
(iii) encoding a second Cholesky matrix with a second Cholesky tensor shape on the first set of qubits, the second set of qubits using the sequence of Multi-controlled gates to form a second Cholesky circuit component, where the control is on the first set of qubits and the data is loaded on ancilla qubits;
(iv) composing a first quantum circuit component by multiplying the first Cholesky circuit component with the first Cholesky tensor shape;
(v) composing a second Cholesky circuit component with the transpose of the second Cholesky tensor shape and the intermediate matrix to obtain sequences of bitstrings;
(vi) computing the Hybrid matrix by diffusing the first Cholesky circuit component and the second Cholesky circuit component with a diffusion operator Rectangular matrix and the Intermediate matrix;
(vii) measuring the second set qubits to obtain all sequences of bitstrings to read entries of the direct matrix; and
(viii) performing amplitude amplification to enhance probability of the sequences of bitstrings by removing overlap between a plurality of states.

7. The Hybrid-Quantum system as claimed in claim 5, wherein the intermediate matrix is computed from the first Cholesky matrix by multiplying the density matrix tensor shape with a rectangular matrix tensor shape.

8. The Hybrid-Quantum system as claimed in claim 5, wherein the Moller-Plesser Perturbations (MP2) energy corrections is performed on the updated density matrix and the Hybrid matrix using the single particle rotation matrix C of the last convergence by performing the steps of:
(i) obtaining the Hybrid matrix, the first set of qubits, the second set of qubits and two copies of single particle unitary rotation matrix (C);
(ii) encoding the first Cholesky tensor shape (Naux, Nao, Nao) with the first set of qubits and the second set of qubits to reshape the first Cholesky tensor;
(iii) loading the reshaped first Cholesky tensor into the quantum circuit;
(iv) repeating the single particle unitary rotation matrix to generate subsets of the first set of qubits;
(v) encoding on the first quantum circuit energy denominator comprising of four single particle Fock energies; and
(vi) obtaining a pair of updated first Cholesky tensor to reiterate the loop to compute the Hybrid matrix.

9. One or more non-transitory machine-readable information storage mediums comprising one or more instructions which when executed by one or more hardware processors cause:
receiving via the one or more classical hardware processors, a chemical compound to extract a plurality of properties;
obtaining via the one or more classical hardware processors, a plurality of atomic coordinates of a plurality of molecular orbitals associated with a plurality of molecules comprised in the chemical compound;
determining via the one or more classical hardware processors, a plurality of inputs from the plurality of atomic coordinates comprising (i) a plurality of electron integrals, (ii) a core Hamiltonian, (ii) a collocation matrix, and (iii) a collocation matrix gradient, wherein the plurality of electron integrals comprises a 3-center 2-electron integrals, 2-center 2-electron integrals, and 2-center 1-electron integrals;
transmitting the plurality of inputs from the one or more classical hardware processors of a classical computer to a one or more quantum processor of a quantum computer;
determining by the one or more quantum processors, a density matrix from the core Hamiltonian;
iteratively performing, by the one or more quantum processors, until a criteria is satisfied, wherein the criteria specifies that a difference between the density matrix and an updated density matrix converges to a value below a threshold convergence error, wherein the steps of each iteration to validate the criteria comprises:
(i) computing, a direct (J) matrix from the density matrix on a second set of qubits and a first set of qubits and loading the direct (J) matrix in a quantum circuit, wherein the quantum circuit comprises a (i) the first set of qubits, the second set of qubits, and a plurality of ancilla qubits;
(ii) computing a Hybrid matrix (K) using (i) a first Cholesky tensor of shape for a first Cholesky circuit component, (ii) a second Cholesky tensor of shape for a second Cholesky circuit component, and (iii) the density matrix encoded with the second set of qubits including one control qubit and one target qubit to store coefficients, wherein Naux is a number of auxillary vectors and Nao is a number of atomic orbitals, wherein in the quantum circuit the first Cholesky circuit component and the first Cholesky circuit component are loaded as inputs;
(iii) determining a correlation exchange matrix based on (i) the collocation matrix using a generalized gradient approximation (GGA), and (ii) the Hybrid (K) matrix energy added for DFT computations;
(iv) computing an Fock matrix based on (i) the core Hamiltonian, (ii) the direct (J) matrix and the Hybrid (K) matrix constructed from the density matrix and the Cholesky tensor, and (iii) the correlation exchange matrix; and
(V) computing an updated density matrix by performing qubitized diagonalization on the Fock matrix and a single particle rotation matrix C; and
performing using the one or more quantum processors, Moller-Plesser Perturbations (MP2) energy corrections on the updated density matrix and the Hybrid (K) matrix using the single particle rotation matrix C of the last convergence when the difference between the density matrix and an updated density matrix converges to a value above the threshold convergence error; and
obtaining from the one or more quantum processors, (i) the quantum circuit, (ii) the updated density matrix, and (iii) the MP2 energy corrections, to be performed by the one or more classical hardware processors for extracting the plurality of properties associated with the chemical compound.

10. The one or more non-transitory machine-readable information storage mediums of claim 9, wherein the Hybrid (K) matrix is computed by performing the steps of:
obtaining the direct (J) matrix and the density matrix;
encoding a first Cholesky matrix on (i) a first set of qubits and (ii) a second set of qubits with a first Cholesky tensor shape using a sequence of Multi-controlled gates to form a Cholesky circuit component, wherein the first Cholesky matrix is created using the 3-center 2-electron integrals, 2-center 2-electron integrals on a CPU, where the control is on the first set of qubits and the data is loaded on the ancilla qubits,
encoding a second Cholesky matrix with a second Cholesky tensor shape on the first set of qubits, the second set of qubits using the sequence of Multi-controlled gates to form a second Cholesky circuit component, where the control is on the first set of qubits and the data is loaded on ancilla qubits;
composing a first quantum circuit component by multiplying the first Cholesky circuit component with the first Cholesky tensor shape;
composing a second Cholesky circuit component with the transpose of the second Cholesky tensor shape and the intermediate matrix to obtain sequences of bitstrings;
computing the Hybrid matrix by diffusing the first Cholesky circuit component and the second Cholesky circuit component with a diffusion operator Rectangular matrix and the Intermediate matrix;
measuring the second set qubits to obtain all sequences of bitstrings to read entries of the direct matrix; and
performing amplitude amplification to enhance probability of the sequences of bitstrings by removing overlap between a plurality of states.

11. The one or more non-transitory machine-readable information storage mediums of claim 9, wherein the intermediate matrix is computed from the first Cholesky matrix by multiplying the density matrix tensor shape with a rectangular matrix tensor shape.

12. The one or more non-transitory machine-readable information storage mediums of claim 9, wherein the MP2 energy corrections are performed on the updated density matrix and the Hybrid matrix using the single particle rotation matrix C of the last convergence by performing the steps of:
obtaining the Hybrid matrix, the first set of qubits, the second set of qubits and two copies of single particle unitary rotation matrix (C);
encoding the first Cholesky tensor shape with the first set of qubits and the second set of qubits to reshape the first Cholesky tensor;
loading the reshaped first Cholesky tensor into the quantum circuit;
repeating the single particle unitary rotation matrix to generate subsets of the first set of qubits;
encoding on the first quantum circuit energy denominator comprising of four single particle Fock energies; and
obtaining a pair of updated first Cholesky tensor to reiterate the loop to compute the Hybrid matrix.
